(19) United States
(12) Patent Application Publication
(10) Pub. No.: US 2020/0246660 A1
(43) Pub. Date: Aug. 6, 2020

(54) REHABILITATION TRAINING SYSTEM AND METHOD USING RGB-D CAMERA

(71) Applicant: UINCARE Corporation, Seoul (KR)

(72) Inventors: Jin Ryoul Kim, Hanam-si (KR); Yong Tae WON, Osan-si (KR)

(21) Appl. No.: 16/279,712

(22) Filed: Feb. 19, 2019

(30) Foreign Application Priority Data
Jan. 31, 2019 (KR) 10-2019-0012997

(51) Int. Cl. A63B 24/00; A61B 5/11; G06K 9/00

(52) U.S. Cl. CPC A63B 24/0006; A61B 5/1128; G16H 20/30; A61B 5/1118; A61B 2505/09; G06K 9/00342

(57) ABSTRACT

A rehabilitation training method using a RGB-D camera is provided, which includes: (a) providing a program screen stored in a database unit to a user through a display unit; (b) receiving program selection information input through a touch type display unit; (c) providing a user information input screen through the display unit and receiving user information input to the information input screen by the user; (d) determining whether to execute the selection program selected by the user in step (b) based on the user information; (e) executing a previous program or the selection program in accordance with the determination of step (d); and (f) providing an evaluation result of the user analyzed by photographing the user who executes the executing program in accordance with the execution of the previous program or the selection program using a RGB-D camera.

### CROSS-REFERENCE TO RELATED APPLICATIONS

This application claims the priority of Korean Patent Application No. 10-2019-0012997 filed on Jan. 31, 2019, in the Korean Intellectual Property Office, the disclosure of which is incorporated herein by reference.

### BACKGROUND

### Field

The present disclosure relates to a rehabilitation training system and method using a RGB-D camera, and more particularly, to a rehabilitation training system and method using a RGB-D camera which photograph a user using a RGB-D camera located in front of the user, display the photographed image in which joints parts of the user are associated with dots, on a display unit in an opposing position, also display a position point of a joint to be located in a correct posture for every specific movement to analyze whether to be agreed with an actual joint point of the user and cause the user to copy the correct posture by moving body parts to perform the rehabilitation training.

### Description of the Related Art

Physical therapy or rehabilitation therapy uses mechanical and instrumental therapy to recover, enhance, and normalize movements and functional physical abilities of patients through massage, functional training, physical correction exercise, and rehabilitation training as much as possible.

However, the above-mentioned physical therapy or rehabilitation therapy is mainly performed in the hospitals rather than at home, so that there is a cumbersome problem in that patients who need the rehabilitation should visit the hospital with uncomfortable body condition, for the therapy.

Further, since the physical therapy or the rehabilitation therapy of the related art is performed hard and dry using the machines or equipment so that it cannot provide fun to the patients. Therefore, there is a problem in that the patient's will to rehabilitate is not strong so that the rehabilitation speed is low, and the efficiency is not high.

### SUMMARY

In order to solve the above-described problem, the present disclosure provides a rehabilitation training system and method using a RGB-D camera which photograph a user using a RGB-D camera located in front of the user, display the photographed image in which joints parts of the user are associated with dots, on a display unit in an opposing position, also display a position point of a joint to be located in a correct posture for every specific movement to analyze whether to be agreed with an actual joint point of the user and cause the user to copy the correct posture by moving body parts to easily perform the rehabilitation training also at home and provide rehabilitation game contents so that the game is played in accordance with the body movement of the user to acquire game points, thereby performing the rehabilitation training by maximizing the will to rehabilitate.

According to an aspect of the present disclosure, a rehabilitation training method using a RGB-D camera includes: (a) providing a program screen stored in a database unit to a user through a display unit, by a main controller of a rehabilitation set top box; (b) receiving program selection information input through a touch type display unit by the main controller; (c) providing a user information input screen through the display unit and receiving user information input to the information input screen by the user, by the rehabilitation set top box; (d) determining whether to execute the selection program selected by the user in step (b) based on the user information, by the main controller; (e) executing a previous program or the selection program in accordance with the determination of step (d), by the main controller; and (f) providing a performance evaluation result of the user analyzed by photographing the user, who performs the execution program in accordance with the execution of the previous program or the selection program, using a RGB-D camera, by the main controller.

In the rehabilitation training method using a RGB-D camera according to the present disclosure, the main controller performs the step (d) including: (d-1) determining whether there is a history that used a rehabilitation training system using a RGB-D camera after receiving the user information in step (c); (d-2) determining whether there is a previous execution program when there is a history that used the rehabilitation training system in step (d-1); and (d-3) confirming whether to continuously execute the previous execution program from the user when there is the previous execution program in step (d-2).

In the rehabilitation training method using a RGB-D camera according to the present disclosure, in the step (e), when there is no history that used the rehabilitation training system using a RGB-D camera in step (d-1), or there is no previous execution program in step (d-2), or it is confirmed that the user does not continuously use the previous execution program in step (d-3), the main controller executes the selection program received in step (b).

In the rehabilitation training method using a RGB-D camera according to the present disclosure, in the step (e), when it is confirmed that the user continuously uses the previous execution program in the step (d-3), the main controller may execute the previous program prior to the selection program.

In the rehabilitation training method using a RGB-D camera according to the present disclosure, when the selection program of the user received in the step (b) is a movement evaluation program, the step (e) includes (e-1) setting a position of a rehabilitation system using a RGB-D camera or guiding the user through the display unit, by the main controller of the rehabilitation set top box; (e-2) uploading an image for performing a training movement to the movement DB to provide the image to the display unit, by the main controller; (e-3) photographing a movement of the user who copies the training movement by the RGB-D camera; (e-4) displaying the image of the user photographed in step (e-3) to associate dots with every joint part on the display unit, by the main controller; (e-5) determining whether movements between the image provided by the main controller and the photographed image are agreed with each other and determining a holding time of the set movement; (e-6) determining correct movement and incorrect movement depending on whether movements are agreed and the holding time is satisfied and counting the number of correct or incorrect movements, by the main controller; (e-7) collecting the counted number of correct or incorrect movements and determining whether the counted number and the number of performances which need to be performed by the user are agreed with each other, by the main controller; and (e-8) repeatedly performing steps after the step (e-5) when the number of correct and incorrect movements and the number of performances are not agreed in step (e-7) and providing a result report including accuracy and completion degree when the numbers are agreed.

In the rehabilitation training method using a RGB-D camera according to the present disclosure, when the selection program of the user received in step (b) is a health training program, the step (e) may include: (e-21) receiving a health training program of a user by the main controller of the rehabilitation set top box; (e-22) providing a sub program corresponding to the health training program stored in the rehabilitation training content DB through the display unit and receiving the received health training program, by the main controller; (e-23) providing a caution for the received health training program to the display unit, by the main controller; (e-24) determining whether the user confirms the caution of the health training program, by the main controller; (e-25) recognizing the user by the RGB-D camera and correcting to a position suitable to execute the health training program when the caution is confirmed, by the main controller; (e-26) explaining an exercising movement for the selected health training program by the main controller; and (e-27) determining whether the user confirms the explanation of the exercising movement and providing a result by receiving and analyzing the exercising movement performed by the user when the explanation is confirmed, by the main controller.

In the rehabilitation training method using a RGB-D camera according to the present disclosure, when the caution of the training program is not confirmed by the user in step (e-24), the main controller repeatedly performs the step (e-23) and when the explanation of the exercising movement of the training program is not confirmed by the user in the step (e-27), repeatedly performs the step (e-26).

In the rehabilitation training method using a RGB-D camera according to the present disclosure, when the selection program of the user received in step (b) is a health game program, the step (e) includes: (e-31) receiving a health game program of a user by the main controller of the rehabilitation set top box; (e-32) providing a plurality of game lists in accordance with the selection of the health game program of the user in step (e-31), by the main controller; (e-33) receiving a game selected by the user from the game list by the main controller; (e-34) providing a game difficulty setting screen for the selected game and game explanation, by the main controller; (e-35) executing the game by reflecting a game setting in accordance with a game start signal input through the display unit, by the main controller; and (e-36) providing a result of the execution game played by the user, by the main controller.

In the rehabilitation training method using a RGB-D camera according to the present disclosure, the main controller may perform a step of recognizing the user located to face the display panel and correcting a posture and a position of the user to a posture or a position appropriate to play the selected game, prior to the step (e-35).

In the rehabilitation training method using a RGB-D camera according to the present disclosure, when the selection program of the user received in the step (b) is a physical strength evaluation program (SFT), the step (e) includes (e-41) providing a demonstration to the display unit, by the main controller; (e-42) suggesting a required condition when the user copies the demonstration, by the main controller; (e-43) photographing a body of the user who copies the demonstration by the RGB-D camera; (e-45) collecting the result by analyzing the body photographed image of the user who copies the demonstration, by the main controller; (e-46) evaluating any one influenced by the demonstration, among a lower strength, an upper strength, a knee joint flexibility, a shoulder joint flexibility, a dynamic balance, and endurance corresponding to the body functions, as the collected result, by the main controller; and (e-47) providing an evaluation result chart, by the main controller.

The rehabilitation training method using a RGB-D camera according to the present disclosure, before the step (e-45), may further include: (e-44) recognizing an evaluation movement by recognizing a body joint photographed by the RGB-D camera and connecting dots which are associated with joint parts of the user to measure the time, an angle, and the number of repetitions, by the main controller.

Further, the rehabilitation training method using a RGB-D camera according to the present disclosure, after the step (e-47), may further include: (e-48) recommending a movement for enhancing a function lack to the user, among a lower strength, an upper strength, a knee joint flexibility, a shoulder joint flexibility, a dynamic balance, and endurance from an exercise database stored as a table, in accordance with an age range and a body part based on the evaluation result chart, by the main controller.

Further, in the rehabilitation training method using a RGB-D camera according to the present disclosure, the main controller may calculate a healthy physical age for male and female from the evaluation result chart by “Y(healthy physical age: male)=45.5+7.4×X₁−2.6×X₂” and “Y(healthy physical age: female)=120+0.1×X₁−5×X₂”.

The rehabilitation training system and method using a RGB-D camera according to the present disclosure allow the rehabilitation at home to solve the inconvenient in that the patients who need the rehabilitation should visit the hospital for the treatment with uncomfortable body condition.

Further, the rehabilitation training system and method using a RGB-D camera according to the present disclosure provide rehabilitation game contents so that the game is played in accordance with the body movements of the user to acquire game points. Therefore, the rehabilitation is performed by maximizing the will to rehabilitate to improve the rehabilitation efficiency.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a view illustrating two examples of rehabilitation training system equipment using a RGB-D camera according to the present disclosure;

FIG. 2 is a specific block diagram of a rehabilitation set top box of a rehabilitation training system using a RGB-D camera according to the present disclosure;

FIG. 3 is a flowchart of a rehabilitation training method using a RGB-D camera according to the present disclosure;

FIG. 4 is a specific flowchart of a step of determining whether to execute a selection program in a rehabilitation training method using a RGB-D camera according to the present disclosure;

FIG. 5 is a specific flowchart of a step of executing a movement evaluation program in a rehabilitation training method using a RGB-D camera according to the present disclosure;

FIG. 6 is a view of a state in which a user performs an actual movement evaluation program according to a rehabilitation training method using a RGB-D camera according to the present disclosure;

FIG. 7 is a view illustrating correct movements and incorrect movements on a graph with blue and red colors;

FIG. 8 is a specific flowchart of a step of performing a health training program in a rehabilitation training method using a RGB-D camera according to the present disclosure;

FIG. 9 is a specific flowchart of a step of performing a health game program in a rehabilitation training method using a RGB-D camera according to the present disclosure;

FIG. 10 is a specific flowchart of a step of performing a physical strength evaluation program (SFT) in a rehabilitation training method using a RGB-D camera according to the present disclosure;

FIG. 11 is an example of a demonstration provided by a physical strength evaluation program (SFT) in a rehabilitation training method using a RGB-D camera according to the present disclosure;

FIG. 12 is a graph of an evaluation result chart after performing a physical strength evaluation program (SFT) in a rehabilitation training method using a RGB-D camera according to the present disclosure;

FIG. 13 is a table for explaining to calculate healthy physical ages for male and female;

FIG. 14 is a table for explaining to calculate physical function ages for male and female; and

FIG. 15 is a view illustrating a structure of a database of exercises recommended by a rehabilitation training method using a RGB-D camera according to the present disclosure which is formed in the form of table.

### DETAILED DESCRIPTION OF THE EMBODIMENT

Those skilled in the art may make various modifications to the present invention and the present invention may have various embodiments thereof, and thus specific embodiments will be described in detail with reference to the drawings. However, it should be understood that the invention is not limited to the specific embodiments, but includes all changes, equivalents, or alternatives which are included in the spirit and technical scope of the present invention. In the description of respective drawings, similar reference numerals designate similar elements.

Terms such as first, second, A, or B may be used to describe various components, but the components are not limited by the above terms. The above terms are used only to discriminate one component from the other component. For example, without departing from the scope of the present invention, a first component may be referred to as a second component, and similarly, a second component may be referred to as a first component. A term of and/or includes a combination of a plurality of related elements or any one of the plurality of related elements.

It should be understood that, when it is described that an element is “coupled” or “connected” to another element, the element may be directly coupled or directly connected to the other element or coupled or connected to the other element through a third element. In contrast, when it is described that an element is “directly coupled” or “directly connected” to another element, it should be understood that no element is present therebetween.

Terms used in the present application are used only to describe a specific exemplary embodiment but are not intended to limit the present invention. A singular form may include a plural form if there is no clearly opposite meaning in the context. In the present application, it should be understood that term “include” or “have” indicates that a feature, a number, a step, an operation, a component, a part or the combination thoseof described in the specification is present, but do not exclude a possibility of presence or addition of one or more other features, numbers, steps, operations, components, parts or combinations, in advance.

If it is not contrarily defined, all terms used herein including technological or scientific terms have the same meaning as those generally understood by a person with ordinary skill in the art. Terms defined in generally used dictionary shall be construed that they have meanings matching those in the context of a related art and shall not be construed in ideal or excessively formal meanings unless they are clearly defined in the present application.

In the specification and the claim, unless explicitly described to the contrary, the word “comprise” and variations such as “comprises” or “comprising”, will be understood to imply the inclusion of stated elements but not the exclusion of any other elements.

Hereinafter, exemplary embodiments according to the present disclosure will be described in detail with reference to accompanying drawings.

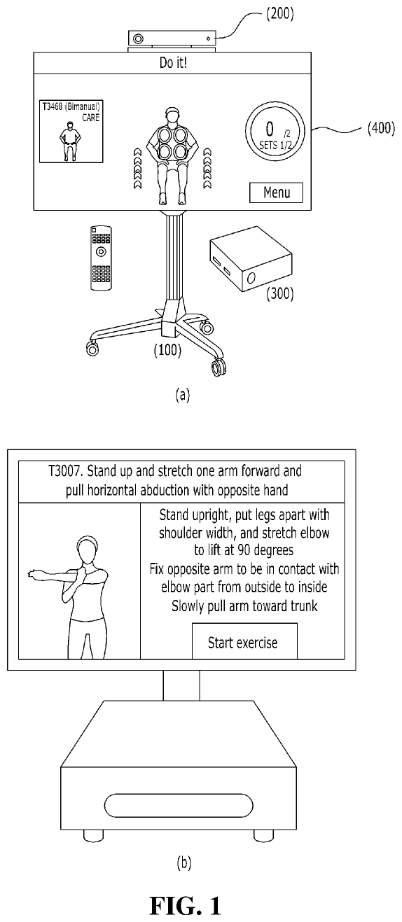

FIG. 1 is a view illustrating rehabilitation training system equipment using a RGB-D camera according to the present disclosure.

As illustrated in FIG. 1A, a rehabilitation training system using a RGB-D camera according to the present disclosure includes a portable stand holder 100, a RGB-D camera 200, a rehabilitation set top box 300, and a display unit 400.

For reference, even though it is illustrated that the RGB-D camera 200 and the rehabilitation set top box 300 of the rehabilitation training system equipment using a RGB-D camera according to the present disclosure are separated in FIG. 1A, as illustrated in FIG. 1B, the RGB-D camera 200 and the rehabilitation set top box 300 may be integrally configured.

Moreover, the rehabilitation training system equipment using a RGB-D camera according to the present disclosure is not limited thereto and the RGB-D camera 200 and the rehabilitation set top box 300 are included in the display unit 400 to be configured as all-in-one type.

The portable stand holder 100 includes a stepping motor therein which rotates by a pulse signal to be movable in a vertical direction by applying pulse signals with different polarities. Therefore, when the user stands in front of the rehabilitation training system equipment using a RGB-D camera according to the present disclosure, the portable stand holder 100 moves up and down in accordance with a height of the user to be set with an appropriate height.

The RGB-D camera 200 is formed above the rehabilitation set top box 300 or the display unit 400 to photograph a user which is opposite thereto.

The rehabilitation set top box 300 deduces an agreement of a movement of the user with a stored standard movement through signal processing for the image photographed by the RGB-D camera to identify a current status of the user and provide a rehabilitation exercise suitable for the current status of the user or provide a game.

The display unit 400 displays the image photographed by the RGB-D camera 200 and a game screen provided by the rehabilitation set top box 300.

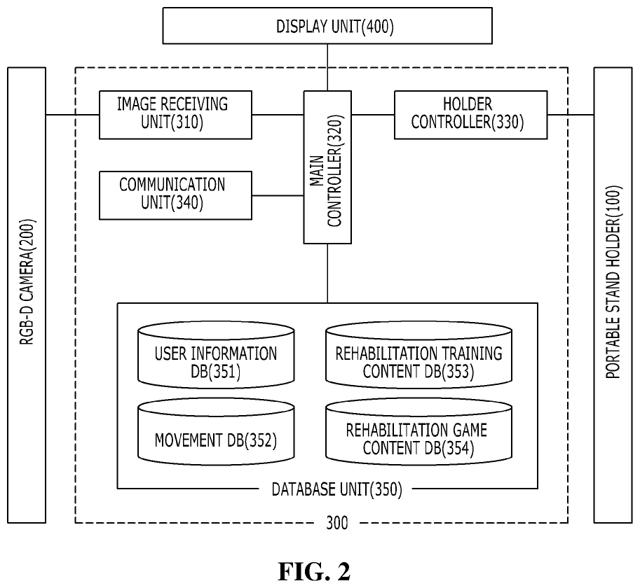

The rehabilitation set top box 300 of the rehabilitation training system using a RGB-D camera according to the present disclosure will be described in more detail with reference to FIG. 2.

As illustrated in FIG. 2, the rehabilitation set top box 300 of the rehabilitation training system using a RGB-D camera according to the present disclosure includes an image receiving unit 310, a main controller 320, a holder controller 330, a communication unit 340, and a database unit 350.

The image receiving unit 310 is connected to the RGB-D camera 200 to receive a three-dimensional image of a user photographed in consideration of a depth by the RGB-D camera 200.

The main controller 320 detects a joint part from the three-dimensional image of the user received by the image receiving unit 310 and associates dots with the detected joint part.

The holder controller 330 is connected to the portable stand holder 100 to move the portable stand holder 100 up and down in accordance with a height of the user which faces the RGB-D camera 200 to set the portable stand holder at an appropriate height.

The communication unit 340 is connected to an Internet communication network to transmit a rehabilitation training result or an evaluation result by the rehabilitation training system using a RGB-D camera according to the present disclosure to a terminal carried by the user or a hospital server so as to be provided to an attending physician of the user.

The database unit 350 includes a user information DB 351, a movement analysis and evaluation DB 352, a rehabilitation training content DB 353, and a rehabilitation game content DB 354.

The user information DB 351 stores and manages all information of the user who uses the rehabilitation training system using a RGB-D camera according to the present disclosure.

The movement analysis and evaluation DB 352 stores a total of 30 types of basic movements such as an upper body, a lower body, and balance which are provided to the user to be copied and stores and manages analysis and evaluation results in accordance with the performance of the movement of the user.

The rehabilitation training content DB 353 stores and manages approximately three hundred types of specialized rehabilitation training contents corresponding to whole body such as shoulder, cervical, lumbar, hip, knee, ankle, elbow, and wrist.

The rehabilitation game content DB 354 stores and manages a plurality of full 3D rehabilitation game training contents designed for a medical use corresponding to various rehabilitation such as the upper body, the lower body, the full body, a balance, ADL, and cognition so as to be utilized for various rehabilitation therapies.

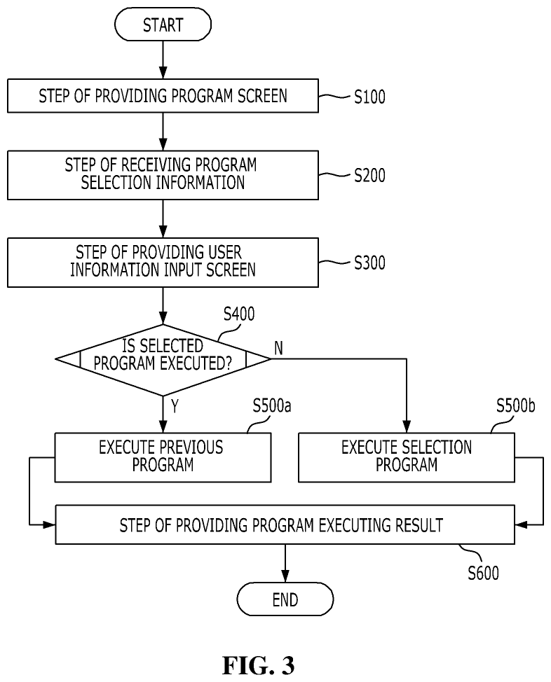

As another exemplary embodiment according to the present disclosure, a rehabilitation training method by the rehabilitation training system using a RGB-D camera having the configuration as described above will be described with reference to FIG. 3.

First, when the user stands in front of the rehabilitation training system using a RGB-D camera according to the present disclosure, the main controller 320 of the rehabilitation set top box 300 performs a step of providing a program screen stored in the database unit 350 to the user through the display unit 400.

More specifically, the main controller 320 provides a program screen to the user through the display unit 400 to allow the user to select any one of a movement evaluation program, a health training program, and a health game program.

The main controller 320 performs a step of receiving program selection information of the user input through the touch type display unit 400 in step S200.

In the meantime, the program selection of the user is not limited to a touch type, but the program may be selected by an input device such as a remote controller.

The rehabilitation set top box 300 performs a step of providing a user information input screen through the display unit 400 and receiving user information input to the information input screen by the user in step S300.

The main controller 320 of the rehabilitation set top box 300 performs a step of determining whether to perform a program selected by the user in step S200 in step S400.

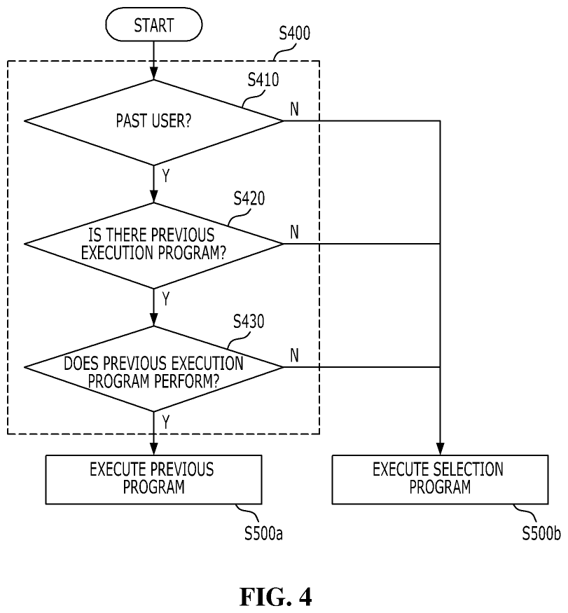

The step S400 of determining whether to perform the program selected by the user will be described in more detail with reference to FIG. 4.

First, the main controller 320 of the rehabilitation set top box 300 performs a step of determining whether there is a history that the rehabilitation training system using a RGB-D camera according to the present disclosure was used in the past, based on the user information received in step S300 in step S410.

When there is no history that the rehabilitation training system using a RGB-D camera according to the present disclosure was used in step S410, the main controller 320 performs a step S500b of executing a program selected by the user in step S200.

When there is a history that the rehabilitation training system using a RGB-D camera according to the present disclosure was used, the main controller 320 performs a step of determining whether there is a previous execution program in step S420.

In step S420, when there is no previous execution program, the main controller 320 performs a step S500b of performing a program selected by the user in step S200.

When there is a previous execution program, the main controller 320 performs a step of determining whether to continuously execute the previous execution program in step S430.

In step S430, when the user continuously executes the previous execution program, the main controller 320 executes the previous program and when the user does not continuously execute the previous execution program, performs the step S500b of executing the program selected by the user in step S200.

As described above, the main controller 320 of the rehabilitation set top box 300 proceeds step S500a of executing the previous program or the step S500b of executing a program selected by the user in step S200, depending on whether to execute the selected program in step S400.

The main controller 320 of the rehabilitation set top box 300 may perform a step of photographing and analyzing the user using the RGB-D camera in accordance with the performance of the step S500a of executing the previous program or the step S500b of executing the selected program to provide the evaluation result to the display unit 400 or to the user terminal or the hospital server through the communication unit 340 in step S600.

Further, after the step S600, the main controller 320 may further perform a step of recommending an exercise suitable for the user, among a plurality of exercises stored in the database unit 350, with the evaluation result and the age information among the user information received in step S300.

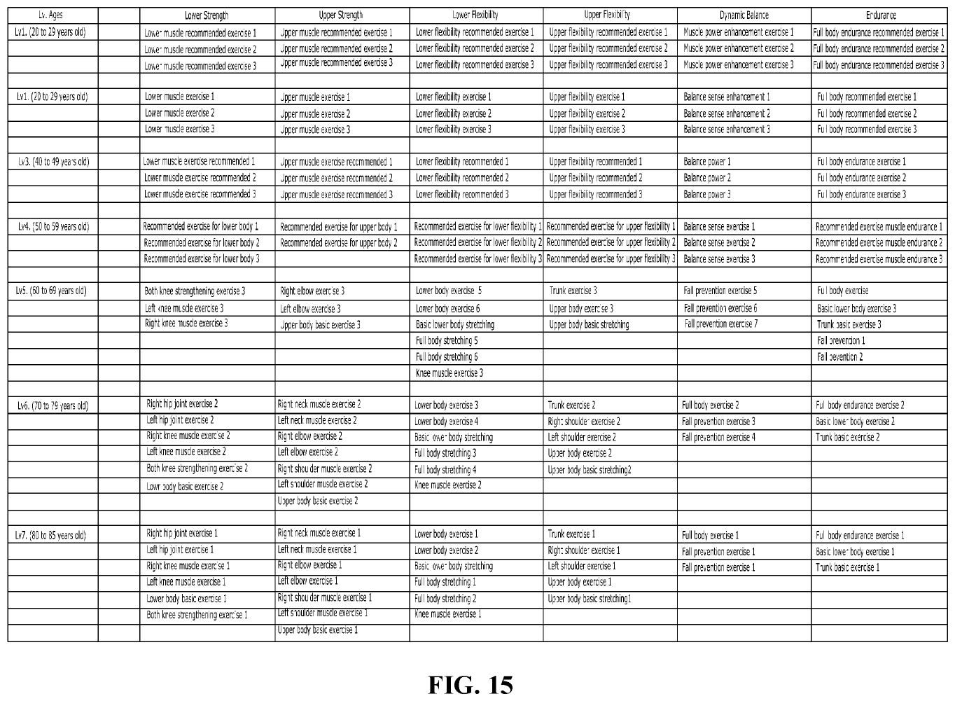

More specifically, in the database unit 350, as illustrated in FIG. 15, an age range in a vertical direction and a body part in a horizontal direction are disposed and different levels or types of recommended exercises for every combined position are stored in a table.

In this case, the main controller 320 recommends the exercise with the evaluation result and the age information. For example, when it is determined that a lower body muscle strength is weak and age information among the user information is seventies as an evaluation result, the main controller 320 may recommend exercises to strengthen a lower body, such as an exercise for a right hip joint, a basic exercise for a lower body, and an exercise for strengthening both knees.

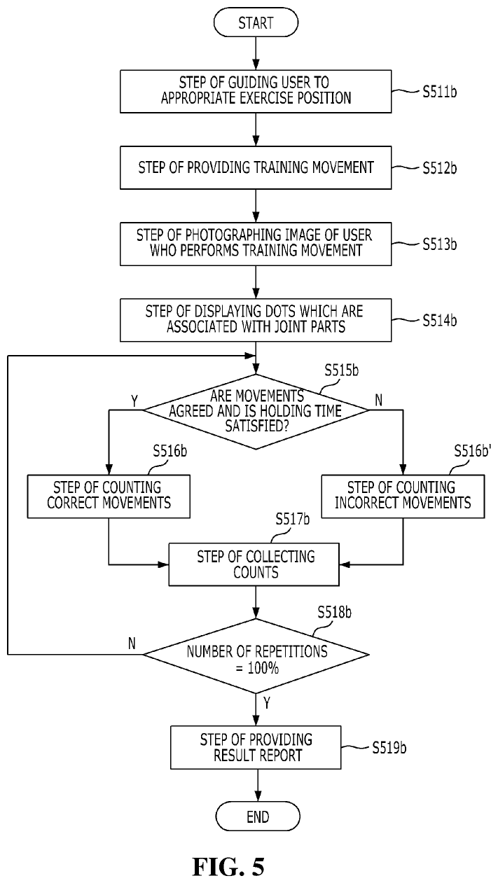

Next, in step S200, when the user selects a movement evaluation program among the movement evaluation program, the health training program, and the health game program in step S200, the step S510b of executing the selected movement evaluation program will be described in detail with reference to FIG. 5.

When the user is located in front of the display unit 400, the main controller 320 of the rehabilitation set top box 300 recognizes the user photographed by the RGB-D camera 200 and identifies the position to control the portable stand holder to perform a step of setting the system in a position appropriate to execute the program or guide the user in step S511b.

When the user is located in a position appropriate to execute the movement evaluation program through the step S511b, the main controller 320 performs a step of providing a learning image stored in the movement DB 352 for performing a correct training movement in step S512b.

In the movement DB 352, a lower body muscle strength evaluation movement which repeatedly sits and stands until the thigh is horizontal to the knees for 15 seconds, an upper body muscle strength evaluation movement which repeatedly bends the elbows and stretches the elbows as much as possible for 15 seconds, a hip joint flexibility evaluation movement which keeps the knees stretched as much as possible until a bell rings and keeps the knees bent as much as possible until the bell rings, a shoulder joint flexibility evaluation movement which keeps the forearms outwardly turned as far as possible until the bell rings and keeps the forearms inwardly turned as far as possible until bell rings, a dynamic balance evaluation movement which goes forward and backward about 2.4 m, and an endurance evaluation movement which steps in place as much as possible for 30 seconds.

In the above step S512b, after providing the training movement, a step of photographing the movement of the user who performs the training movement by the RGB-D camera 200 is performed in step S513b.

The main controller 320 performs a step of displaying the image in which the dots are associated with the joint parts of the user, photographed by the RGB-D camera 200, on the display unit in an opposite position in step S514d.

As described above, in step S514b, the dots are associated with the joint parts of the user so as to allow the user to precisely perform the training movement of the user and allow the main controller 320 to more precisely analyze the training movement of the user.

The main controller 320 of rehabilitation set top box 300 performs a step of determining the agreement of the movements and a holding time in step S515b.

That is, in step S515b, the main controller 320 determines whether the performed movement is correct depending on whether the movement provided in step S512b and the movement performed by the user in step S513b are agreed with each other and/or whether the set holding time is satisfied.

In other words, the main controller 320 determines whether the performed movement is correct with the agreement of movements or the holding time set by software.

When the performed movement satisfies the agreement of the movements and/or the set holding time in step S515b, the main controller 320 performs a step of counting as “correct” in step s516b and when the performed movement does not satisfy the agreement of movements and/or the set holding time, the main controller 320 performs a step of counting as “incorrect” in step S516b′.

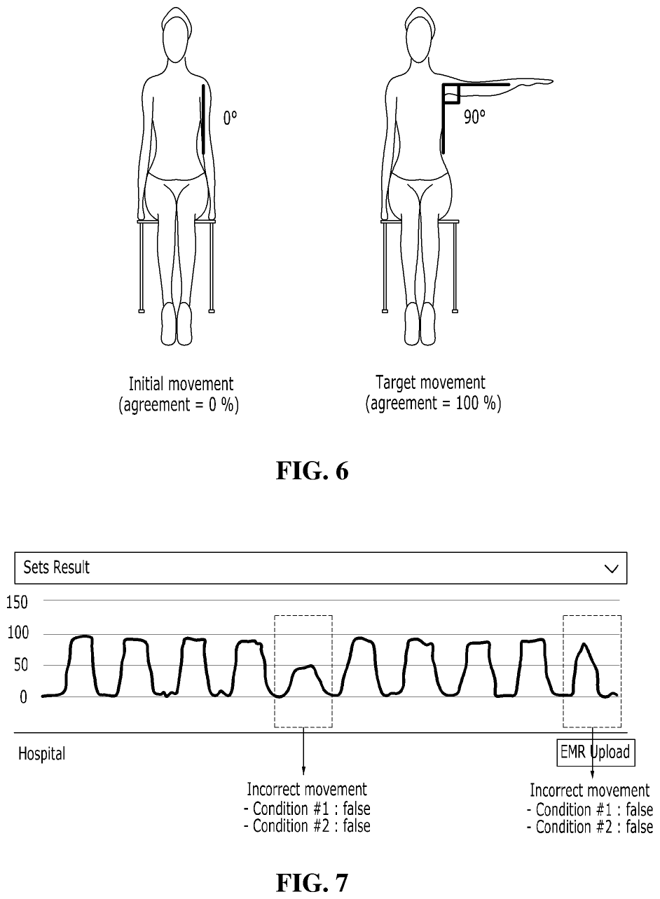

For example, as illustrated in FIG. 6, when a training movement 1 which abducts the shoulders to 90° (shoulder abduction to 90°) is performed, when a measured angle of the shoulder joint is 0° based on a target joint angle 90° set using the following Equation 1, the main controller 320 calculates the agreement as 0% and when the measured angle is 90°, calculates the agreement as 100%.

$$Agreement of movement ( Unit \text{:} \% ) = \frac{Measurement angle}{Target angle of joint} \times 100 \qquad [ Equation 1 ]$$

As another example, when a training movement 2 which abducts the hip to 35° is performed, the main controller 320 calculates the agreement using the Equation 1 and determines whether it is a correct movement by applying the agreement (>80%).

For reference, the settings were made depending on the judgments of medical teams which participate in the software development.

The main control unit 320 determines whether to be correct movement depending on whether the first condition (agreement) is satisfied and the movement is kept for a holding time (an exercise performing setting value in the program and default is 2 seconds) as a set second condition.

As illustrated in FIG. 7, a graph in accordance with the agreement of movement is represented in a training result report and a blue graph indicates a correct movement and a red graph indicates an incorrect movement.

The main controller 320 performs a step of collecting all counting numbers in step S517 from the correct movement counting step S516b and the incorrect movement counting step S516b′ and performs a step S518b of determining whether the collected counting numbers match the number of performances which need to be performed by the user to perform a step of providing a result report when the collected counting numbers match the number of performances in step S519b.

When the counting numbers do not match the number of performances to be performed by the user in step S518b, the main controller 320 repeats the step S515b until the counting numbers match the number of performances to be performed by the user.

In the meantime, in step S519b, the main controller 320 represents the number of correct movements as a percentage by comparing the number of correct movements with a total repetition in the report and denotes the accuracy as “accuracy” [performance metrics: unicare_protocol_result_Accuracy] in the result report and calculates the accuracy using the following Equation 2.

$$Accuracy ( unit \text{:} \% ) = \frac{The number of correct movement}{Total repetition} \times 100 \qquad [ Equation 2 ]$$

Further, the main controller 320 denotes a movement completion of the user through the total number of correct/incorrect movements as “Completion” [performance metrics; uincare_protocol_result_Completion] and calculates the movement completion using the following Equation 3.

$$Completion ( unit \text{:} \% ) = \frac{[ \begin{matrix}The number of correct movement + \\ the number of incorrect movement\end{matrix} ]}{Total repetition} \times 100 \qquad [ Equation 3 ]$$

After the step S519b, the main controller 320 may further include a step of recommending exercises in accordance with the result report.

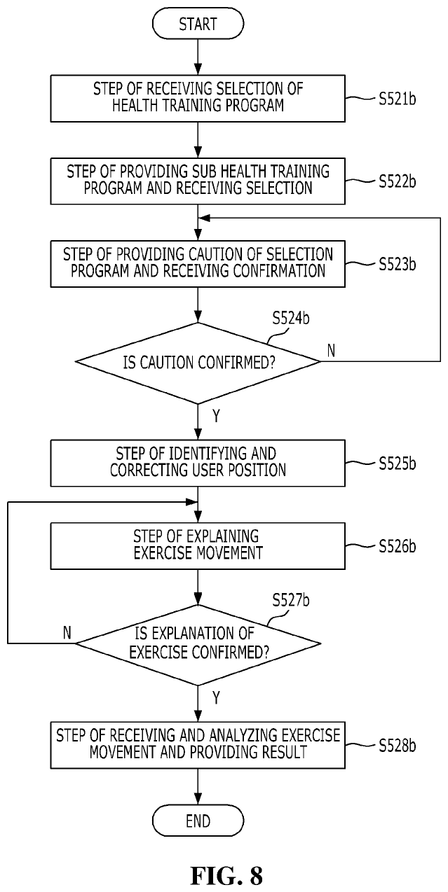

Next, in step S200, when the user selects the health training program among the movement evaluation program, the health training program, and the health game program, the step S520b of executing the selected health training program will be described in detail with reference to FIG. 8.

In the meantime, a description of the contents described with respect to the above-described step of executing the movement evaluation program will be omitted in the following description.

The main controller 320 of the rehabilitation set top box 300 performs a step of receiving the selection of the health training program of the user in step S521b.

The main controller 320 performs a step of providing a selection screen for the muscle strength, the flexibility, the balance, and the endurance health training program stored in the rehabilitation training content DB 353 to the display unit 400 and receiving the selected training program input by the touch of the user through the display unit 400 in step S522b.

The main controller 320 performs a step of providing cautions for the selected health training program to the display unit 400 in step S523b.

Thereafter, the main controller 320 performs a step S524b of determining whether the user confirms the cautions of the health training program and when the user confirms the cautions, the main controller 320 performs a step of recognizing the user facing the display unit 400 and correcting the position in step S525b.

In step S524b, when the user does not confirm the cautions of the training program, the main controller 320 repeatedly performs the step S523b of providing the cautions.

The main controller 320 performs a step of explaining an exercise movement for the selected health training program in step S526b.

Thereafter, the main controller 320 performs a step S527b of determining whether the user confirms the explanation of the exercise movement of the training program and when the user confirms the explanation, the main controller 320 performs a step of receiving and analyzing the exercise movement performed by the user and providing a result in step S528b.

In step S527b, when the user does not confirm the explanation of the exercise movement of the training program, the main controller 320 repeatedly performs the step S526b of explaining the exercise movement.

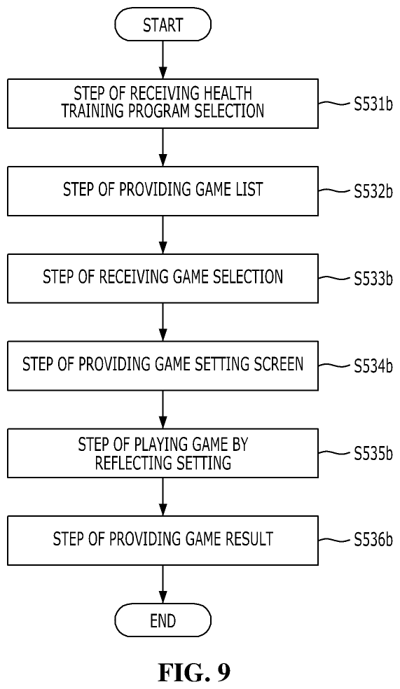

Finally, in step S200, when the user selects the health game program among the movement evaluation program, the health training program, and the health game program in step S200, the step S530b of executing the selected health game program will be described in detail with reference to FIG. 9.

The main controller 320 of the rehabilitation set top box 300 performs a step of receiving the selection of the health game program of the user in step S531b.

The main controller 320 performs a step of providing a plurality of game lists in accordance with the selection of the health game program of the user in step S531 in step S532b.

More specifically, the main controller 320 provides a health training game (racing, go-away and go-away, stepping in place, crash-crash, catch-catch) and cognition games (memory pictures, match pairs, memory patterns, quick touch, and chasing ball).

The main controller 320 performs a step of receiving the game selected from the provided game lists by the user in step S533b and a step of providing a game difficulty setting screen for the selected game and explanation of the game to the user in step S534b.

The main controller 320 performs a step of playing the game by reflecting the game setting in accordance with a game start signal input through the display unit 400 in step S535b.

In this case, the main controller 320 may perform first a step of recognizing the user located to face the display and correcting the position or a posture, prior to the game playing step S535b.

The main controller performs a step of providing a game result such as an exercising time, game points, and a success rate after the user ends the game in step S536b.

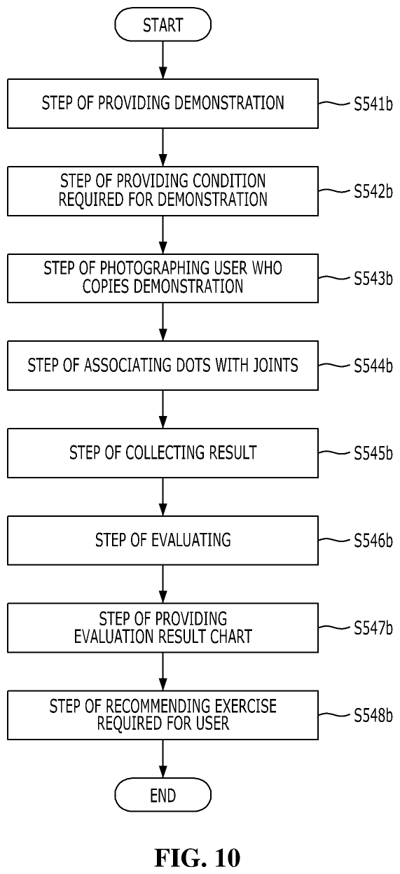

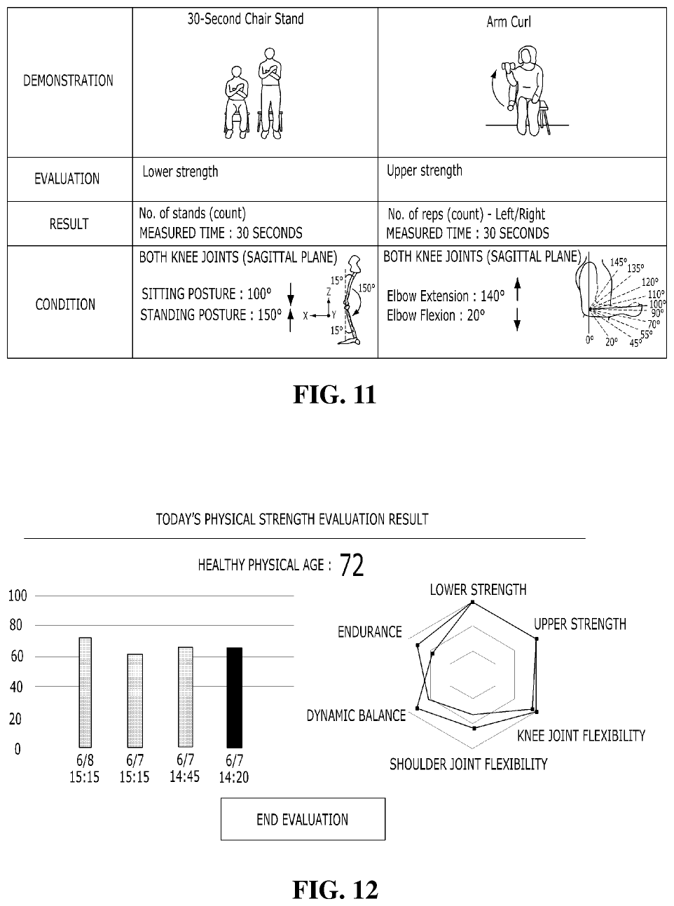

Additionally, in step S200, a step S540b of performing a physical strength evaluation program (SFT) for the elderly will be described in more detail with reference to FIGS. 10 and 11.

FIG. 10 is a specific flowchart of a step of performing a physical strength evaluation program (SFT) in a rehabilitation training method using a RGB-D camera according to the present disclosure.

FIG. 11 is an example of a demonstration provided by a physical strength evaluation program (SFT) in a rehabilitation training method using a RGB-D camera according to the present disclosure.

The main controller 320 performs a step of providing a demonstration for evaluating six functions of the elderly, that is, a lower body strength, an upper body strength, a knee joint flexibility, a shoulder joint flexibility, a dynamic balance, and endurance in step S541b.

Simultaneously, the main controller 320 performs a step of suggesting a condition required when the user copies the demonstration, on the display unit, in step S542b.

The RGB-D camera 200 performs a step of photographing the body of the user who copies the demonstration in accordance with the condition in step S543.

In order to recognize the evaluation movement by recognizing the body joint photographed by the RGB-D camera 200 in step S543 and easily measure the time, an angle, and the number of times, the main controller 320 performs a step of connecting dots which are associated with the joint parts of the user in the photographed image in step S544b.

The main controller 320 performs a step of collecting a result that the user copies the demonstration in step S545b and then performs a step of evaluating any one of six physical functions evaluated by the demonstration in step S546.

For example, as illustrated in the left side of FIG. 11, steps of providing standing on a chair for 30 seconds as a demonstration in step S541b, suggesting a condition that both knee joints are 100° or lower in a sitting posture and are 150° or higher in a standing posture in step S542b, photographing the user who copies the demonstration in accordance with the condition, by the RGB-D camera 200, in step S543b, and receiving the images to collect a result of calculating the number of repetitions of sitting and standing for 30 seconds in step S545b, and then evaluating a lower strength among six physical functions influenced by the demonstration in step S546 are performed.

Similarly, a step of providing arm turning illustrated in the right side of FIG. 11 as a demonstration, suggesting a condition that the elbow stretches to 140° or more and is bent to 20° or less, collecting the result of counting the number of turning of arms for 30 seconds, and then evaluating an upper strength among six physical functions influenced by the arm turning movement.

After the evaluating step S546, as illustrated in FIG. 12, a step of providing an evaluation result chart as illustrated in FIG. 12 is performed in step S547.

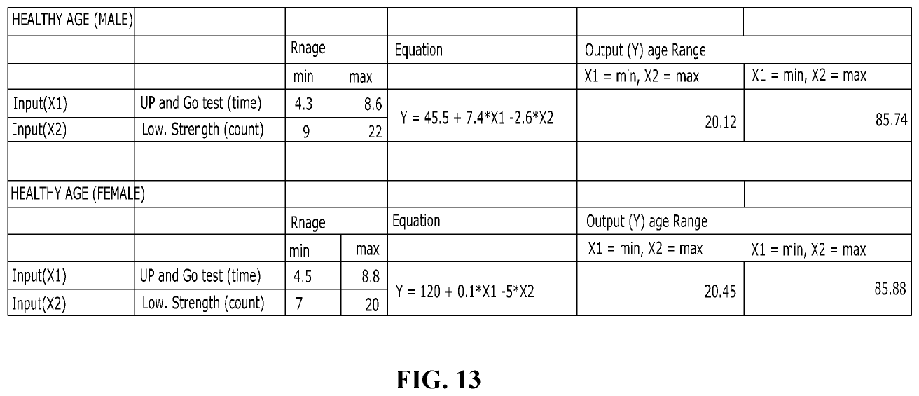

The main controller 320 distinguishes healthy physical ages in the evaluation result chart illustrated in FIG. 12 for male and female as illustrated in FIG. 13 to calculate the result for age range using measurement values of the lower strength and the dynamic balance as inputs of X1 and X₂ of the following Equation 4.

Y(Healthy physical age: male)=45.5+7.4*X₁−2.6*X₂

Y(Healthy physical age: female)=120+0.1*X₁−5*X₂ [Equation 4]

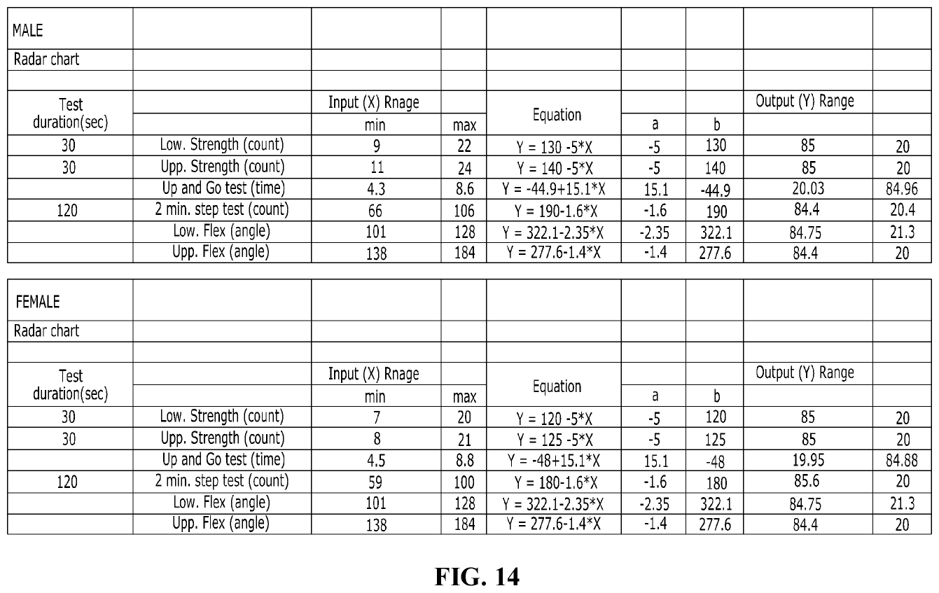

The main controller 320 distinguishes the age ranges for six functions in the evaluation result chart illustrated in FIG. 12 for male and female and uses the measurement value as inputs of FIG. 14 to convert the measurement value into a percentage (%) to display (for example, 20 years old->100%, 85 years old->50%).

The main controller 320 performs a step of recommending an exercise required for the user from a database in which as illustrated in FIG. 15, six functions including a lower strength, an upper strength, a knee joint flexibility, a shoulder joint flexibility, a dynamic balance, and endurance are stored in the table, based on the evaluation result chart illustrated in FIG. 12, in step S548b.

That is, the main controller 320 selects and recommends an exercise from a database in which an age range is disposed in a vertical direction and a body part is disposed in a horizontal direction and a different level or type of recommended exercises for every combined position are stored in a table.

It will be appreciated that various exemplary embodiments of the present invention have been described herein for purposes of illustration, and that various modifications, changes, and substitutions may be made by those skilled in the art without departing from the scope and spirit of the present invention. Therefore, the exemplary embodiments of the present disclosure are provided for illustrative purposes only but not intended to limit the technical concept of the present disclosure. The scope of the technical concept of the present disclosure is not limited thereto. The protective scope of the present disclosure should be construed based on the following claims, and all the technical concepts in the equivalent scope thereof should be construed as falling within the scope of the present disclosure.

What is claimed is:

1. A rehabilitation training method using a RGB-D camera, comprising:
(a) providing a program screen stored in a database unit to a user through a display unit, by a main controller of a rehabilitation set top box;
(b) receiving program selection information input through a touch type display unit by the main controller;
(c) providing a user information input screen through the display unit and receiving user information input to the information input screen by the user, by the rehabilitation set top box;
(d) determining whether to execute the selection program selected by the user in step (b) based on the user information, by the main controller;
(e) executing a previous program or the selection program in accordance with the determination of step (d), by the main controller; and
(f) providing a performance evaluation result of the user analyzed by photographing the user, who performs the execution program in accordance with the execution of the previous program or the selection program, using a RGB-D camera, by the main controller.

2. The rehabilitation training method according to claim 1, wherein the main controller performs the step (d) including:
(d-1) determining whether there is a history that used a rehabilitation training system using a RGB-D camera after receiving the user information in step (c);
(d-2) determining whether there is a previous execution program when there is a history that used the rehabilitation training system in step (d-1); and
(d-3) confirming whether to continuously execute the previous execution program from the user when there is a previous execution program in step (d-2).

3. The rehabilitation training method according to claim 2, wherein in step (e), when there is no history that used the rehabilitation training system using a RGB-D camera in step (d-1), or there is no previous execution program in step (d-2), or it is confirmed that the user does not continuously use the previous execution program in step (d-3), the main controller executes the selection program received in step (b).

4. The rehabilitation training method according to claim 2, wherein in the step (e), when it is confirmed that the user continuously uses the previous execution program in step (d-3), the main controller executes the previous program prior to the selection program.

5. The rehabilitation training method according to claim 3, wherein when the selection program of the user received in step (b) is a movement evaluation program, the step (e) includes:
(e-1) setting a position of a rehabilitation system using a RGB-D camera or guiding the user through the display unit, by the main controller of the rehabilitation set top box;
(e-2) uploading an image for performing a training movement to the movement DB to provide the image to the display unit, by the main controller;
(e-3) photographing a movement of the user who copies the training movement by the RGB-D camera;
(e-4) displaying the image of the user photographed in step (e-3) to associate dots with every joint part on the display unit, by the main controller;
(e-5) determining whether movements between the image provided by the main controller and the photographed image are agreed with each other and determining a holding time of the set movement;
(e-6) determining correct movement and incorrect movement depending on whether movements are agreed and the holding time is satisfied and counting the number of correct or incorrect movements, by the main controller;
(e-7) collecting the counted number of correct or incorrect movements and determining whether the counted number and a number of performances which need to be performed by the user are agreed with each other, by the main controller; and
(e-8) repeatedly performing steps after step (e-5) when the number of correct and incorrect movements and the number of performances are not agreed in step (e-7) and providing a result report including accuracy and completion degree when the numbers are agreed.

6. The rehabilitation training method according to claim 3, wherein when the selection program of the user received in step (b) is a health training program, step (e) includes,
(e-21) receiving a health training program of a user by the main controller of the rehabilitation set top box;
(e-22) providing a sub program corresponding to the health training program stored in the rehabilitation training content DB through the display unit and receiving the received health training program, by the main controller;
(e-23) providing a caution for the received health training program to the display unit, by the main controller;
(e-24) determining whether the user confirms the caution of the health training program, by the main controller;
(e-25) recognizing the user by the RGB-D camera and correcting to a position suitable to execute the health training program when the caution is confirmed, by the main controller;
(e-26) explaining an exercising movement for the selected health training program by the main controller; and
(e-27) determining whether the user confirms the explanation of the exercising movement and providing a result by receiving and analyzing the exercising movement performed by the user when the explanation is confirmed, by the main controller.

7. The rehabilitation training method according to claim 6, wherein when the caution of the training program is not confirmed by the user in step (e-24), the main controller repeatedly performs step (e-23) and when the explanation of the exercising movement of the training program is not confirmed by the user in step (e-27), repeatedly performs step (e-26).

8. The rehabilitation training method according to claim 3, wherein when the selection program of the user received in step (b) is a health game program, the step (e) includes,
(e-31) receiving a health game program of a user by the main controller of the rehabilitation set top box;
(e-32) providing a plurality of game lists in accordance with the selection of the health game program of the user in step (e-31), by the main controller;
(e-33) receiving a game selected by the user from the game list by the main controller;
(e-34) providing a game difficulty setting screen for the selected game and game explanation, by the main controller;
(e-35) executing the game by reflecting a game setting in accordance with a game start signal input through the display unit, by the main controller; and
(e-36) providing a result of the execution game played by the user, by the main controller.

9. The rehabilitation training method according to claim 3, wherein the main controller performs: a step of recognizing the user located to face the display unit and correcting a posture and a position of the user to a posture or a position appropriate to play the selected game, prior to step (e-35).

10. The rehabilitation training method according to claim 3, wherein when the selection program of the user received in step (b) is a physical strength evaluation program (SFT), step (e) includes:
(e-41) providing a demonstration to the display unit, by the main controller;
(e-42) suggesting a required condition when the user copies the demonstration, by the main controller;
(e-43) photographing a body of the user who copies the demonstration by the RGB-D camera;
(e-45) analyzing the body photographed image of the user who copies the demonstration and collecting the result, by the main controller;
(e-46) evaluating any one influenced by the demonstration, among a lower strength, an upper strength, a knee joint flexibility, a shoulder joint flexibility, a dynamic balance, and endurance corresponding to the body functions, as the collected result, by the main controller; and
(e-47) providing an evaluation result chart, by the main controller.

11. The rehabilitation training method according to claim 10, further comprising: before step (e-45),
(e-44) recognizing an evaluation movement by recognizing a body joint photographed by the RGB-D camera and connecting dots which are associated with joint parts of the user to measure the time, an angle, and the number of repetitions, by the main controller.

12. The rehabilitation training method according to claim 11, further comprising: after step (e-47),
(e-48) recommending a movement for enhancing a function lack to the user, among a lower strength, an upper strength, a knee joint flexibility, a shoulder joint flexibility, a dynamic balance, and endurance from an exercise database stored as a table, in accordance with an age range and a body part based on the evaluation result chart, by the main controller.

13. The rehabilitation training method according to claim 12, wherein the main controller calculates a healthy physical age for male and female from the evaluation result chart by “Y (healthy physical age: male)=45.5+7.4×X₁−2.6×X₂” and “Y (healthy physical age: female)=120+0.1×X₁−5×X₂”, respectively, and X₁ is a measurement value of low strength and X₂ is a measurement value of a dynamic balance.
